(19) United States
(12) Patent Application Publication
(10) Pub. No.: US 2014/0073643 A1
(43) Pub. Date: Mar. 13, 2014

(54) TREATMENT OF JAK2-MEDIATED CONDITIONS

(71) Applicants: Gregg D. Smith, Balaclava (AU); Rose Fida, Middle Park (AU); Mark M. Kowalski, Winchester, MA (US)

(72) Inventors: Gregg D. Smith, Balaclava (AU); Rose Fida, Middle Park (AU); Mark M. Kowalski, Winchester, MA (US)

(73) Assignee: YM BIOSCIENCES AUSTRALIA PTY LTD, Mount Eliza (AU)

(21) Appl. No.: 13/991,139

(22) PCT Filed: Nov. 29, 2011
(86) PCT No.: PCT/AU2011/001551

§ 371 Date: Nov. 15, 2013

Related U.S. Application Data
(60) Provisional application No. 61/419,476, filed on Dec. 3, 2010.
(60) Provisional application No. 61/492,485, filed on Jun. 2, 2011.

(51) Int. Cl. A61K 31/5377

(52) U.S. Cl. CPC A61K 31/5377
USPC 514/235.8

(57) ABSTRACT

Described herein is a method for treating a subject experiencing anemia, comprising administering to the subject an amount of N-(cyanomethyl)-4-[2-[[4-(4-morpholinyl)phenyl]amino]-4-pyrimidinyl]-benzamide [CYT387 ] or a related compound effective to maintain or elevate the hemoglobin level. The anemic subjects including those afflicted with a myeloproliferative disorder, such as primary myelofibrosis.

### FIELD OF THE INVENTION

This invention relates to the enzyme Janus kinase 2, or JAK2. More particularly, the invention relates to the use of JAK2 inhibitors in the treatment of myeloproliferative neoplasms and related disorders, as well as associated conditions including anemia.

### BACKGROUND TO THE INVENTION

JAKs are kinases which phosphorylate a group of proteins called Signal Transduction and Activators of Transcription or STATs. When phosphorylated, STATs dimerize, translocate to the nucleus and activate expression of genes which lead to, amongst other things, cellular proliferation.

The central role played by the JAK family of protein tyrosine kinases in the cytokine dependent regulation of both proliferation and end function of several important cell types indicates that agents capable of inhibiting the JAK kinases are useful in the prevention and chemotherapeutic treatment of disease states dependent on these enzymes. Potent and specific inhibitors of each of the currently known four JAK family members will provide a means of inhibiting the action of the cytokines that drive immunological and inflammatory diseases.

Myeloproliferative disorders (MPD) include, among others, polycythemia vera (PV), primary myelofibrosis (PMF), thrombocythemia, essential thrombocythemia (ET), idiopathic myelofibrosis (IMF), chronic myelogenous leukemia (CML), systemic mastocystosis (SM), chronic neutrophilic leukemia (CNL), myelodysplastic syndrome (MDS) and systemic mast cell disease (SMCD). JAK2 is a member of the JAK family of kinases in which a specific mutation (JAK2V617F) has been found in 99% of polycythemia vera (PV) patients and about 50% of essential thrombocytopenia (ET) and idiopathic myelofibrosis (MF). This mutation is thought to activate JAK2, giving weight to the proposition that a JAK2 inhibitor will be useful in treating these types of diseases.

Primary myelofibrosis is a particularly devastating disease that afflicts patients that are typically older than 65 years and is marked by lower than normal hemoglobin levels, and an elevated level of white cells and circulating blasts.

A number of JAK inhibitors are currently in clinical development for the treatment of MPD. These include INCB018424 for the treatment of primary myelofibrosis, XL019, SB 1518 and AZD1480 for the treatment of post-PV/ET myelofibrosis, and TG101348 for treatment of JAK2V617F-positive ET. As well, the phenylamino pyrimidine designated CYT387 is the subject of on-going clinical trials for the treatment of primary myelofibrosis and post-polycythemia vera myelofibrosis and post-essential thrombocythemia myelofibrosis.

It is an object of the present invention to provide a method useful for the treatment of subjects afflicted with myeloproliferative neoplasms, such as primary myelofibrosis.

It is a further object of the present invention to provide a method useful to maintain or to elevate hemoglobin levels in subjects experiencing anemia, including subjects afflicted with myeloproliferative neoplasms and other blood disorders or cancers.

### SUMMARY OF THE INVENTION

It has now been found that the effects of CYT387 therapy are particularly pronounced in myeloproliferative disease patients that meet certain criteria. Patients that meet one or more of these criteria can thus now be identified and then recruited for CYT387 therapy, in accordance with the present invention. These patients benefit particularly from a pronounced anemia response and/or improved spleen response, both of which are clinical indicators of JAK inhibitor drug efficacy.

The pronounced effect of CYT387 on hemoglobin levels, notable as an anemia response, indicates that this compound is useful to promote either a maintenance or elevation of hemoglobin levels in subjects that are anemic. The anemic subjects include subjects that are hemoglobin deficient as a result of a blood disorder such as a blood cancer, including myeloproliferative disease, as a result of chemotherapy treatment with anti-neoplastic agents or modalities, or as a result of other medical conditions that affect the level of functioning red blood cells.

In one aspect of the present invention, there is provided a method for treating an anemic subject comprising administering to the subject an amount of CYT387 effective to maintain or elevate the level of hemoglobin in that subject. In a related aspect, the invention provides for the use of CYT387 for the treatment of anemia. In embodiments, the subject is an anemic subject afflicted with a myeloproliferative neoplasm such as primary myelofibrosis, as well as myelofibrosis secondary to polycythemia vera (PV) or essential thrombocythemia (ET). In other embodiments, the subject is an anemic subject afflicted with myelodysplastic syndrome (MDS).

In one aspect, the invention provides an improved method for treating a subject having, or at risk for, a medical condition for which a JAK inhibitor such as a JAK1/2 inhibitor or a JAK2 inhibitor is indicated, the method comprising the steps of (1) selecting, for treatment, a subject that presents with, or is at risk for, myeloproliferative disease and meets at least one of the following criteria:
(i) prior therapy with a drug selected from thalidomide, lenalidomide, pomalidomide, and a JAK inhibitor such as a JAK inhibitor other than CYT387;
(ii) a clinical criterion selected from one or both of (1) enlarged spleen size and 2) a lower percentage of circulating blasts;
(iii) a biochemical marker criterion selected from one or more of (1) an increased level of at least one protein selected from EGF, TNF-α, G-CSF, IFN-α, MIP-1β, HGF, MIG, and VEGF; (2) a decreased level of eotaxin; and (3) an altered level of at least one protein selected from EPO, hepcidin and BMP-2;
   and then (2) administering to the selected subject a treatment-effective amount of CYT387, whereby the treated subject exhibits an anemia response and/or a spleen response that is improved relative to a subject not meeting at least one of said criteria.

In a related aspect, the present method comprises the step of assessing the subject or a biological sample obtained therefrom, identifying a subject meeting at least one of the criteria noted above, and then treating the identified subject with CYT387. Similarly, the present method also comprises the step of assessing the subject or a blood sample obtained therefrom to identify a subject experiencing anemia, and then treating the identified subject with an amount of CYT387 effective to maintain or elevate the endogenous level of hemoglobin.

In embodiments, the subject is afflicted with primary myelofibrosis. In other embodiments, the subject is afflicted with myelofibrosis secondary to polycythemia vera (PV) or essential thrombocythemia (ET). In a further embodiment, the subject is afflicted with MDS.

In still other embodiments, subjects experiencing anemia can be subjects afflicted with primary myelofibrosis or PV or ET, and can also be subjects experiencing anemia as a result of a broader scope of blood disorders including blood cancers, or as a result of treatment with a chemotherapeutic agent, or as a result of anemia of chronic disease or other causes of anemia.

In another embodiment, the selected subject is a transfusion dependent subject. In a further embodiment, the transfusion dependent subject is treated with CYT387 or a related compound using a dosing regimen that results in, and preferably sustains, transfusion independence.

In another aspect of the present invention, there is provided an article of manufacture, comprising CYT387 in combination with a label indicating treatment of a subject presenting with at least one of the noted criteria, including anemia.

In a related aspect of the present invention, there is provided a kit comprising CYT387 in combination with printed instruction teaching a method of selecting a subject for CYT387 therapy based on the selection criteria herein described, including anemia.

Embodiments of the invention are now described in greater detail with reference to the accompanying Figures in which:

### BRIEF REFERENCE TO THE FIGURES

FIG. 1 shows graphically the effect of CYT387 on hemoglobin levels in patients with primary myelofibrosis, post-PV myelofibrosis or post ET myelofibrosis enrolled in the dose escalation and dose confirmation phases of an ongoing Phase I/II clinical study (All patients; N=60). It also shows the effect of CYT387 on hemoglobin levels in the combined subgroup of these patients with baseline hemoglobin levels of <10 g/dL and those who are transfusion dependent at baseline. This subgroup would be considered to have a marked anemia at baseline.

FIG. 2 shows graphically the effect of CYT387 on hemoglobin levels in three subgroups of patients with primary myelofibrosis, post-PV myelofibrosis or post ET myelofibrosis enrolled into the dose escalation and dose confirmation phases of an ongoing Phase I/II clinical study. These subgroups include (1) all patients who required frequent transfusions of red blood cells (RBC) prior to enrolment [Tx dependent at baseline] (2) the subgroup of transfusion dependent patients who responded to treatment with CYT387 by becoming transfusion independent [Tx responders] and (3) the subgroup of transfusion dependent patients who did not achieve a sufficient response to CYT387 to qualify as responders [Tx non-responders].

### DETAILED DESCRIPTION OF THE INVENTION

CYT387 is a phenylaminopyrimidine compound having CAS registration number CAS 1056634-68-4, the chemical name N-(cyanomethyl)-4-[2-[[4-(4-morpholinyl)phenyl]amino]-4-pyrimidinyl]-benzamide, and the structure shown below:

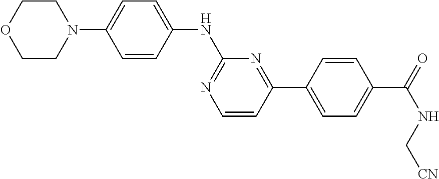 [SMILES: N#CCNC(=O)c1ccc(-c2ccnc(Nc3ccc(N4CCOCC4)cc3)n2)cc1]

Synthesis, formulation and therapeutic use of CYT387 is described in WO 2008/109943 published 18 Sep. 2008; and in Blood, 2010, 115(25):5232-40. Of course, CYT387 can be used in the form of a salt, solvate or prodrug if desired.

In addition to CYT387, the present method can be practiced using structural analogs of CYT387 that share its kinase-binding profile or signature, compounds that are referred to herein as “related compounds”.

“Related compounds” are compounds related to CYT387 by their selective JAK inhibition signature, in which a preference is shown for binding to and inhibition of JAK2 and JAK1, relative to JAK3 and other members of the kinase family, and by their structural conformance to the formula:

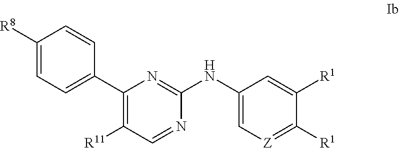

wherein
Z is independently selected from N and CH;
R¹ is independently selected from H, halogen, OH, CONHR₂, CON(R₂)2, CF₃, R²OR², CN, morpholino, thiomorpholinyl, thiomorpholino-1,1-dioxide, substituted or unsubstituted piperidinyl, substituted or unsubstituted piperazinyl, imidazolyl, substituted or unsubstituted pyrrolidinyl and C₁₋₄alkylene wherein the carbon atoms are optionally replaced with NR^{Y} and/or O substituted with morpholino, thiomorpholinyl, thiomorpholino-1,1-dioxide, substituted or unsubstituted piperidinyl, substituted or unsubstituted piperazinyl, imidazolyl or substituted or unsubstituted pyrrolidinyl;
R² is substituted or unsubstituted C₁₋₄alkyl;
R^{Y} is H or substituted or unsubstituted C₁₋₄alkyl;

### R⁸ is R^{X}CN;

R^{X} is substituted or unsubstituted C₁₋₄alkylene wherein up to 2 carbon atoms can be optionally replaced with CO, NSO₂R¹, NR^{Y}, CONR^{Y}, SO, SO₂ or O;
R¹¹ is H, halogen, C1-4alkyl or C₁₋₄alkyloxy, or an enantiomer thereof, a prodrug thereof or a pharmaceutically acceptable salt thereof.

The term “C₁₋₄alkyl” refers to straight chain or branched chain hydrocarbon groups having from 1 to 4 carbon atoms. Examples include methyl, ethyl, propyl, isopropyl, butyl, isobutyl, sec-butyl, and tert-butyl.

The term “halogen” refers to fluorine, chlorine, bromine and iodine.

The term “substituted” refers to a group that is substituted with one or more groups selected from C₁₋₄ alkyl, C₃₋₆ cycloalkyl, C₂₋₆ alkenyl, C₂₋₆ alkynyl, C₁₋₆ alkylaryl, aryl, heterocycylyl, halo, haloC₁₋₆alkyl, haloC₃₋₆cycloalkyl, haloC₂₋₆alkenyl, haloC₂₋₆alkynyl, haloaryl, haloheterocycylyl, hydroxy, C₁₋₆ alkoxy, C₂₋₆alkenyloxy, C₂₋₆ alkynyloxy, aryloxy, heterocyclyloxy, carboxy, haloC₁₋₆alkoxy, haloC₂₋₆alkenyloxy, haloC₂₋₆alkynyloxy, haloaryloxy, nitro, nitroC₁₋₆, alkyl, nitroC₂₋₆alkenyl, nitroaryl, nitroheterocyclyl, azido, amino, C₁₋₆alkylamino, C₂₋₆alkenylamino, C₂₋₆alkynylamino, arylamino, heterocyclamino acyl, C₁₋₆alkylacyl, C₂₋₆alkenylacyl,

C₂₋₆alkynylacyl, arylacyl, heterocycylylacyl, acylamino, acyloxy, aldehydro, C₁₋₆alkylsulphonyl, arylsulphonyl, C₁₋₆alkylsulphonylamino, arylsulphonylamino, C₁₋₆alkylsulphonyloxy, arylsulphonyloxy, C₁₋₆alkylsulphenyl, C₂₋₆ alklysulphenyl, arylsulphenyl, carboalkoxy, carboaryloxy, mercapto, C₁₋₆alkylthio, arylthio, acylthio, cyano and the like.

Preferred substituents are selected from the group consisting of C₁₋₄ alkyl, C₃₋₆ cycloalkyl, C₂₋₆ alkenyl, C₂₋₆ alkynyl, C₁₋₆ alkylaryl, aryl, heterocycylyl, halo, haloaryl, haloheterocycylyl, hydroxy, C₁₋₄ alkoxy, aryloxy, carboxy, amino, C1-6 alkylacyl, arylacyl, heterocycylylacyl, acylamino, acyloxy, C₁₋₆alkylsulphenyl, arylsulphonyl and cyano.

The term “aryl” refers to single, polynuclear, conjugated or fused residues of aromatic hydrocarbons. Examples include phenyl, biphenyl, terphenyl, quaterphenyl, naphthyl, tetrahydronaphthyl, anthracenyl, dihydroanthracenyl, benzanthracenyl, dibenxanthracenyl and phenanthrenyl.

The term “unsaturated N-containing 5 or 6-membered heterocyclyl” refers to unsaturated, cyclic hydrocarbon groups containing at least one nitrogen. Suitable N-containing heterocyclic groups include unsaturated 5 to 6-membered heteromonocyclic groups containing 1 to 4 nitrogen atoms, for example, pyrrolyl, pyrrolinyl, imidazolyl, pyrazolyl, pyridyl, pyrimidinyl, pyrazinyl, pyridazinyl, triazolyl or tetrazolyl; unsaturated 5 or 6-membered heteromonocyclic group containing 1 to 2 oxygen atoms and 1 to 3 nitrogen atoms, such as, oxazolyl, isoxazolyl or oxadiazolyl; and unsaturated 5 or 6-membered heteromonocyclic group containing 1 to 2 sulphur atoms and 1 to 3 nitrogen atoms, such as, thiazolyl or thiadiazolyl.

In preferred embodiments, compounds related to CYT387 include those in which R¹ is substituted in the para position by morpholinyl and in the ortho position by H, Z is carbon, and R¹¹ is H, halogen, methyl or methoxy.

In particularly preferred embodiments, R⁸ is —C(O)—NH—CH₂—CN; —C(O)—NH—C(CH₃)₂CN; or —NH—C(O)—CH₂—CN.

Specific compounds related to CYT387 useful in accordance with the present method include:
N-(cyanomethyl)-4-(2-(4-morpholinophenylamino)pyrimidin-4-yl)benzamide;
N-(cyanomethyl)-3-(2-(4-morpholinophenylamino)pyrimidin-4-yl)benzamide;
N-(cyanomethyl)-3-methyl-4-(2-(4-morpholinophenylamino)pyrimidin-4-yl)benzamide;
N-(cyanomethyl)-2-methyl-4-(2-(4-morpholinophenylamino)pyrimidin-4-yl)benzamide;
2-cyano-N-(3-(2-(4-morpholinophenylamino)pyrimidin-4-yl)benzyl)acetamide;
2-cyano-N-(3-(2-(4-morpholinophenylamino)pyrimidin-4-yl)phenyl)acetamide;
N-(cyanomethyl)-4-(2-(3-morpholinophenylamino)pyrimidin-4-yl)benzamide;
N-(cyanomethyl)-4-(2-(4-thiomorpholinophenylamino)pyrimidin-4-yl)benzamide;
N-(cyanomethyl)-4-(2-(4-(morpholinomethyl)phenylamino)pyrimidin-4-yl)benzamide.
4-(5-chloro-2-((4-morpholinophenyl)amino)pyrimidin-4-yl)-N-(cyanomethyl)benzamide;
4-(5-bromo-2-((4-morpholinophenyl)amino)pyrimidin-4-yl)-N-(cyanomethyl)benzamide;
N-(cyanomethyl)-4-(2-((4-(4-hydroxypiperidin-1-yl)phenyl)amino)pyrimidin-4-yl)benzamide; and
N-(cyanomethyl)-4-(5-methyl-2-((4-morpholinophenyl)amino)pyrimidin-4-yl)benzamide

In the present method, CYT387 or a related compound is used to maintain or elevate hemoglobin levels in a subject experiencing anemia or a hemoglobin decline. Anemic subjects have an endogenous hemoglobin level that is lower than the level that is normal for a healthy subject of equivalent age and gender. Acceptable or “normal” levels are now well established in medical practice. For an adult human male, anemia is evident when the hemoglobin level is below about 13.0 g/dL; for non-pregnant adult human females, deficiency is evident when the hemoglobin level is below about 12.0 g/dL. Measurement of hemoglobin levels is performed using well established techniques. Conditions of severe anemia are evident when the hemoglobin level is less than about 8.0 g/dL.

In use, CYT387 or a related compound is administered to an anemic subject in an amount effective to maintain or elevate the level of hemoglobin in the subject. Administration of the drug thus has the minimum effect of inhibiting further reduction in the level of hemoglobin in the treated subject. More desirably, administration of the drug has the effect of increasing the level of hemoglobin in the subject.

Anemic subjects that would benefit from treatment with CYT387 or a related compound include subjects that have undergone or are undergoing chemotherapy or radiation therapy, such as cancer patients. A wide variety of chemotherapeutic agents are known to have the consequence of reducing the level of functioning red blood cells. As well, subjects that are CYT387 treatment candidates are those afflicted with blood disorders including blood cancers that result in, or are associated with, a reduction in red blood cell count. In embodiments, the subjects to be treated are subjects having anemia associated with or resulting from such blood conditions as myelodysplastic syndrome. Myelodysplastic syndromes (MDS) is a term used to describe a group of diseases characterized by ineffective hematopoiesis leading to blood cytopenias and hypercellular bone marrow. MDS has traditionally been considered to be synonymous with ‘preleukemia’ because of the increased risk of transformation into acute myelogenous leukemia (AML). Evolution to AML and the clinical consequences of cytopenias are main causes of morbidity and mortality in MDS. Debilitating symptoms of MDS include fatigue, pallor, infection, and bleeding. Anemia, neutropenia, and thrombocytopenia are also common clinical manifestations of MDS. In other embodiments, the subjects to be treated are subjects having anemia associated with or resulting from such other blood conditions as anemias associated with other hematologic malignancies, aplastic anemia, anemia of chronic disease that affect red blood cells and the like. Anemia of chronic disease is associated with such diseases as certain cancers including lymphomas and Hodgkin's disease; autoimmune diseases such as rheumatoid arthritis, systemic lupus erythematosis, inflammatory bowel disease and polymyalgia rheumatica; long term infections such as urinary tract infection, HIV and osteomyelitis; heart failure; and chronic kidney disease. In addition, patients with anemia resulting from conditions associated with increased destruction, shortened red blood cell survival and splenic sequestration could also benefit from CYT387 treatment. Patients afflicted with these conditions thus can be treated to improve upon their state of declining or deficient hemoglobin.

In certain embodiments, the subject to be treated is an anemic subject experiencing thalassemia. In other embodiments, the subject to be treated is a subject other than a subject experiencing thalassemia.

In embodiments, CYT387 or a related compound is administered to a subject diagnosed with a myeloproliferative disease such as myeloproliferative neoplasm, thereby to improve upon the prognosis of the disease and, in embodiments, particularly to treat hemoglobin deficiency or decline associated with the disease. In other embodiments, CYT387 or a related compound is administered to an anemic subject that is other than an anemic subject diagnosed with a myeloproliferative disease. This class of treatable subject presents with anemia unrelated to myeloproliferative disease.

“Myeloproliferative diseases” and “myeloproliferative neoplasms (MPN)” most notably polycythemia vera (PV), essential thrombocythemia (ET) and primary myelofibrosis (PMF) are a diverse but inter-related group of clonal disorders of pluripotent hematopoietic stem cells that share a range of biological, pathological and clinical features including the relative overproduction of one or more cells of myeloid origin, growth factor independent colony formation in vitro, marrow hypercellularity, extramedullary hematopoiesis, spleno- and hepatomegaly, and thrombotic and/or hemorrhagic diathesis. An international working group for myeloproliferative neoplasms research and treatment (IWG-MRT) has been established to delineate and define these conditions (see for instance Vannucchi et al, CA Cancer J. Clin., 2009, 59:171-191), and those disease definitions are to be applied for purposes of this specification.

Subjects, most notably human patients, who present with MPN and particularly PMF are identifiable in the art using the IWG-MRT criteria mentioned above. Subjects “at risk for” a particular form of MPN are subjects having an early stage form of the disease, and may for instance include subjects having a genetic marker thereof, such as the JAK2V617F allele which is associated with PV (>95%), with ET (60%) and with PMF (60%). Subjects are also considered to be “at risk for” a form of MFN if they already manifest symptoms of an earlier stage form. Thus, subjects presenting with MFN are at risk for post-PV and post-ET, both of which develop following MPN.

The response of MPN patients and particularly PMF patients to CYT387 therapy is particularly robust when, according to the present invention, they are patients selected for CYT387 therapy based on one or more of the following criteria:

(i) prior therapy with a drug selected from thalidomide, lenalidomide, pomalidomide and a JAK2 inhibitor other than CYT387;

(ii) a clinical criterion selected from one or both of (1) smaller spleen size and (2) a lower percentage of circulating blasts;

(iv) a biochemical marker criterion selected from one or more of (1) an increased level of at least one protein selected from EGF, TNF-α, G-CSF, IFN-α, MIP-1β, HGF, MIG, and VEGF; (2) a decreased level of eotaxin; and (3) an altered level of at least one protein selected from EPO, hepcidin and BMP-2;

The improved outcome from CYT387 therapy that results from prior patient selection is manifest as a robust improvement in anemia response and/or in spleen response.

By “anemia response” is meant an increase in the patient's hemoglobin level or a patient who was transfusion dependent becoming transfusion independent. Desirably, a minimum increase in hemoglobin of 2.0 g/dL lasting a minimum of 8 weeks is achieved, which is the level of improvement specified in the International Working Group (IWG) consensus criteria. However, smaller, but still medically significant, increases in hemoglobin are also considered to be within the term “anemia response”.

By “spleen response” is meant a reduction in the size of the patient's spleen as assessed by either palpation of a previously palpable spleen during physical exam or by diagnostic imaging. The IWG consensus criteria specifies that there be either a minimum 50% reduction in palpable splenomegaly (spleen enlargement) of a spleen that is at least 10 cm at baseline (prior to treatment) or of a spleen that is palpable at more than 5 cm at baseline becomes not palpable. However, smaller reductions are also considered to be within the term “spleen response”.

In one embodiment, the selected patient is one that has received prior drug therapy. More particularly, patients selected for CYT387 therapy include patients that have been treated, or are currently being treated, with thalidomide (CAS number 50-35-1) or with a derivative thereof, particularly lenalidomide (CAS number 191732-72-6). These drugs are both used in the treatment of multiple myeloma, and appear also to be showing some benefit in patients afflicted with myeloproliferative disorder. To receive the further benefit resulting from subsequent CYT387 therapy, patients will either be undergoing treatment with thalidomide, lenalidomide or pomalidomide or similar agent or will have been treated with one of these drugs within a time frame, relative to CYT387 therapy onset, sufficient for the effects of these drugs to be manifest. Patients meeting these criteria experience significant anemia response, relative to patients naïve to this drug therapy, when subsequently treated with CYT387. In a preferred embodiment, the CYT387 patient is one subjected to prior therapy with lenalidomide.

Patients selected for CYT387 therapy also include patients that have been treated, or are undergoing treatment, with a JAK inhibitor other than CYT387. It has been found in particular that patients previously treated with the JAK inhibitor designated INCB018424, or the JAK inhibitor designated TG101348, have a more prominent spleen response to CYT387 therapy than patients naïve to such prior therapy. In a preferred embodiment, the patient selected for CYT387 therapy is one that, in addition to being subjected to therapy with a JAK inhibitor other than CYT387, is also a transfusion dependent patient. INCB018424 is administered at starting doses of 15 or 20 mg po BID with dose titration from 5 mg BID to 25 mg BID. TG101348 is administered once a day with a maximum tolerated dose (MTD) determined to be 680 mg/day. JAK inhibitors other than CYT387 include all and any other JAK inhibitors, and particularly other JAK inhibitors having a JAK affinity, selectivity or binding site different from CYT387. These properties can be determined using the JAK2 crystal structure and the modeling approach and activity assays described in U.S. Pat. No. 7,593,820, the entire disclosure of which is incorporated herein by reference. To receive the further benefit resulting from subsequent CYT387 therapy, patients will either be undergoing treatment with the other JAK2 inhibitor or will have been treated with such a drug within a time frame, relative to CYT387 therapy onset, sufficient for the effects of that JAK2 inhibitor to be manifest in the patient.

Patients selected for CYT387 therapy also include patients having altered levels of detectable protein markers. More particularly, patients in whom the levels of certain protein markers, including certain cytokines and chemokines, are elevated can experience significant benefit when treated with CYT387, in terms of their anemia response and/or their spleen response to CYT387 therapy. In embodiments, elevation in the level of one or more of the following protein markers signifies that the patient is a preferred candidate for CYT387 therapy:

(1) EGF, or epidermal growth factor, the mature form of which comprises residues 971-1023 of the sequence having Swiss-Prot designation P01133;
(2) TNF-α, or tumour necrosis factor alpha, the mature and soluble form of which comprises residues 77-233 of the sequence having Swiss-Prot designation P01375;
(3) G-CSF, or granulocyte colony stimulating factor, the mature form of which comprises residues 30-207 of the sequence having Swiss-Prot designation P09919;
(4) IFN-α, or interferon alpha, comprises a family of subtypes the mature forms of which are well known in the art;
(5) MIP-1β, or macrophage inflammatory protein 1-beta (now known also as C—C motif chemokine 4, or CCL4), the mature form which comprises either residues 24-92 or 26-92 of the sequence having Swiss-Prot designation P13236;
(6) HGF, or hepatocyte growth factor, the mature forms of which are based on the sequence having Swiss-Prot designation P14210, and include the alpha chain having residues 32-494 and the beta chain having residues 495-728;
(7) MIG, or monokine induced by gamma interferon (now known also as CXCL9), is within the family of chemotactic cytokines, the mature form of which comprises residues 23-125 of the sequence having Swiss-Prot designation Q07325;
(8) VEGF, or vascular endothelial growth factor A, the mature form of which comprises residues 27-232 of the sequence having Swiss-Prot designation P15692.

Patients presenting for CYT387 therapy experience a significant spleen response when they are selected initially based on an elevation in the level of any one or more of the markers noted above. An elevated level is a level that is greater than the level in a normal subject.

Patients presenting for CYT387 therapy can also experience a significant anemia response when they are selected initially based on a depression in the level of the protein eotaxin. This protein, known also as eosinophil chemotatic protein and comprising residues 24-97 of the sequence having Swiss-Prot designation P51671, functions through interaction with CCR3 to promote accumulation of esoinophils in response to allergens, a prominent feature of allergic inflammatory reactions.

Still other markers useful to select patients for CYT387 therapy include altered levels of EPO, hepcidin and BMP-2.

The “level” of a given marker is considered to be altered, i.e., either elevated or reduced, when the level measured in a given patient is different to a statistically significant extent from the corresponding level in a normal subject. Patients that present with marker levels altered to an extent sufficient, desirably, to yield a p value of at least 0.05 or more significant, i.e., better, are selected as candidates for CYT387 therapy. In embodiments, the p value is at least 0.03, 0.02 or 0.01, and in preferred embodiments the p value is at least 0.009, 0.007, 0.005, 0.003, 0.001 or better.

The levels of a given marker can be determined using assays already well established for detection the markers noted above. In embodiments, this is achieved by extracting a biological sample from the patient candidate, such as a sample of whole blood or a fraction thereof such as plasma or serum. The sample then is treated to enrich for the marker of interest, if desired, and the enriched or neat sample is assayed for instance using a detectable ligand for the marker, such as a labeled antibody that binds selectively to the marker. The amount of marker present in the sample can then be determined either semi-quantitatively or quantitatively, to obtain a value that is then compared against a reference value that is the normal level for that marker in a healthy subject. As noted above, a difference in marker levels sufficient to arrive at a p value that is at least 0.05 indicates an altered marker level of significance, and patients presenting with an elevated level of that marker (or in the case of eotaxin, a decreased level) are candidates for CYT387 therapy.

Also suitable as candidates for CYT387 therapy are those patients that meet certain clinical criteria, including those presenting with a spleen of relatively small size, and those presenting with an elevated level of circulating, or peripheral, blasts. These patients respond to CYT387 therapy particularly well, in terms of their spleen response. In one embodiment, the selected patient is one that has not yet progressed to transfusion dependency. Splenic enlargement is assessed by palpation. Splenic size and volume can also be measured by diagnostic imaging such as ultrasound, CT or MRI). Normal spleen size is approximately 11.0 cm. in craniocaudal length.

Also suitable as candidates for CYT387 therapy are those patients presenting with a lower percentage of circulating blasts. Blasts are immature precursor cells that are normally found in the bone marrow and not the peripheral blood. They normally give rise to mature blood cells. The lower percentage of circulating blasts is measured by cytomorphologic analysis of a peripheral blood smear as well as multiparameter flow cytometry and immunohistochemistry. As a prognostic factor >/=1% blasts is used.

The present invention also provides both an article of manufacture and a kit, comprising a container comprising CYT387 in an amount effective to treat MPN. The container may be simply a bottle comprising CYT387 in oral dosage form, each dosage form comprising a unit dose of CYT387, in an amount for instance from about 50 mg to 400 mg, such as 150 mg, 200 mg or 300 mg. The kit will further comprise printed instructions teaching the present method of selecting subjects for CYT387. The article of manufacture will comprise a label or the like, indicating treatment of a subject according to the present method of patient selection.

The present invention also provides both an article of manufacture and a kit, comprising a container comprising CYT387 in an amount effective to treat anemia. The container may be simply a bottle comprising CYT387 in oral dosage form, each dosage form comprising a unit dose of CYT387, in an amount for instance from about 50 mg to 400 mg, such as 150 mg, 200 mg or 300 mg. The kit will further comprise printed instructions teaching the present method of selecting anemic subjects for CYT387 therapy. The article of manufacture will comprise a label or the like, indicating treatment of an anemic subject.

For use in the present method, CYT387 or a related compound is formulated according to standard pharmaceutical practice.

The compounds may be prepared as salts which are pharmaceutically acceptable, such as salts of pharmaceutically acceptable cations such as sodium, potassium, lithium, calcium, magnesium, ammonium and alkylammonium; acid addition salts of pharmaceutically acceptable inorganic acids such as hydrochloric, orthophosphoric, sulfuric, phosphoric, nitric, carbonic, boric, sulfamic and hydrobromic acids; or salts of pharmaceutically acceptable organic acids such as acetic, propionic, butyric, tartaric, maleic, hydroxymaleic, fumaric, citric, lactic, mucic, gluconic, benzoic, succinic, oxalic, phenylacetic, methanesulfonic, trihalomethanesulfonic, toluenesulfonic, benzenesulfonic, isethionic, salicylic, sulphanilic, aspartic, glutamic, edetic, stearic, palmitic, oleic, lauric, pantothenic, tannic, ascorbic, valeric and orotic acids. Salts of amine groups may also comprise quaternary ammonium salts in which the amino nitrogen atom carries a suitable organic group such as an alkyl, alkenyl, alkynyl or aralkyl moiety.

In one embodiment, the compound is a hydrochloride salt of CYT387, such as a dihydrochloride salt of CYT387.

Where a compound possesses a chiral center the compound can be used as a purified enantiomer or diastereomer, or as a mixture of any ratio of stereoisomers. It is however preferred that the mixture comprises at least 70%, 80%, 90%, 95%, 97.5% or 99% of the preferred isomer, where the preferred isomer gives the desired level of potency and selectivity.

Prodrugs of CYT387 and related compounds can also be administered. For example, compounds having free amino, amido, hydroxy or carboxylic acid groups can be converted into prodrugs. Prodrugs include compounds wherein an amino acid residue, or a polypeptide chain of two or more (e.g., two, three or four) amino acid residues which are covalently joined through peptide bonds to free amino, hydroxy and carboxylic acid groups of compounds of the invention. The amino acid residues include the 20 naturally occurring amino acids commonly designated by three letter symbols and also include, 4-hydroxyproline, hydroxylysine, demosine, isodemosine, 3-methylhistidine, norvlin, beta-alanine, gamma-aminobutyric acid, citrulline, homocysteine, homoserine, ornithine and methioine sulfone. Prodrugs also include compounds wherein carbonates, carbamates, amides and alkyl esters which are covalently bonded to a substituent of the drug compound.

The compound is administered as a pharmaceutical composition comprising the compound and a pharmaceutically acceptable carrier. The carrier must be “pharmaceutically acceptable” means that it is compatible with the other ingredients of the composition and is not deleterious to a subject. The compositions may contain other therapeutic agents as described below, and may be formulated, for example, by employing conventional solid or liquid vehicles or diluents, as well as pharmaceutical additives of a type appropriate to the mode of desired administration (for example, excipients, binders, preservatives, stabilizers, flavours, etc.) according to techniques such as those well known in the art of pharmaceutical formulation (See, for example, Remington: The Science and Practice of Pharmacy, 21st Ed., 2005, Lippincott Williams & Wilkins).

The compound may be administered by any suitable means, for example, orally, such as in the form of tablets, capsules, granules or powders; sublingually; buccally; parenterally, such as by subcutaneous, intravenous, intramuscular, intra(trans)dermal, or intracisternal injection or infusion techniques (e.g., as sterile injectable aqueous or non-aqueous solutions or suspensions); nasally such as by inhalation spray or insufflation; topically, such as in the form of a cream or ointment ocularly in the form of a solution or suspension; vaginally in the form of pessaries, tampons or creams; or rectally such as in the form of suppositories; in dosage unit formulations containing non-toxic, pharmaceutically acceptable vehicles or diluents. The compounds may, for example, be administered in a form suitable for immediate release or extended release. Immediate release or extended release may be achieved by the use of suitable pharmaceutical compositions comprising the compounds, or, particularly in the case of extended release, by the use of devices such as subcutaneous implants or osmotic pumps.

The pharmaceutical compositions for the administration may conveniently be presented in dosage unit form and may be prepared by any of the methods well known in the art of pharmacy. These methods generally include the step of bringing the compound of formula I into association with the carrier which constitutes one or more accessory ingredients. In general, the pharmaceutical compositions are prepared by uniformly and intimately bringing the compound into association with a liquid carrier or a finely divided solid carrier or both, and then, if necessary, shaping the product into the desired formulation. In the pharmaceutical composition the compound is included in an amount sufficient to produce the desired effect upon the process or condition of diseases. When delivered orally and once daily, a unit dose lies preferably in the range from 50 mg to 300 mg. This amount is effective to maintain or elevate hemoglobin levels in anemic recipients.

The pharmaceutical compositions is desirably in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents such as sweetening agents, flavouring agents, colouring agents and preserving agents, e.g. to provide pharmaceutically stable and palatable preparations. Tablets contain the compound of formula I in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated to form osmotic therapeutic tablets for control release.

Formulations for oral use may also be presented as hard gelatin capsules wherein the compound is mixed with an inert solid diluent, for example, calcium carbonate, calcium phosphate or kaolin, or as soft gelatin capsules wherein the compound is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the compound in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the compound in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative, and flavoring and coloring agents.

The pharmaceutical compositions may be in the form of a sterile injectable aqueous or oleaginous suspension. This suspension may be formulated according to the known art using those suitable dispersing or wetting agents and suspending agents which have been mentioned above. The sterile injectable preparation may also be a sterile injectable solution or suspension in a non-toxic parenterally acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectable formulations.

The compound may also be presented for use in the form of veterinary compositions, which may be prepared, for example, by methods that are conventional in the art. Examples of such veterinary compositions include those adapted for:

(a) oral administration, external application, for example drenches (e.g. aqueous or non-aqueous solutions or suspensions); tablets or boluses; powders, granules or pellets for admixture with feed stuffs; pastes for application to the tongue;
(b) parenteral administration for example by subcutaneous, intramuscular or intravenous injection, e.g. as a sterile solution or suspension; or (when appropriate) by intramammary injection where a suspension or solution is introduced in the udder via the teat;
(c) topical applications, e.g. as a cream, ointment or spray applied to the skin; or
(d) rectally or intravaginally, e.g. as a pessary, cream or foam.

In the treatment of an identified subject, an appropriate unit dose of the selected drug compound will generally be about 0.01 to 500 mg per kg patient body weight per day which can be administered in single or multiple doses. The dosage level will be about 0.1 to about 250 mg/kg per day; such as about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. Suitable unit doses will typically be in the range from 10 to 500 mgs, such as 50-400 mgs, e.g., 100, 150, 200, 250 or 300 mgs. For oral administration, the compositions are preferably provided in the form of tablets containing 1.0 to 1000 milligrams of the active ingredient, particularly 1, 5, 10, 15, 20, 25, 50, 75, 100, 150, 200, 250, 300, 400, 500, 600, 750, 800, 900, and 1000 milligrams of the active ingredient. The dosage may be selected, for example to any dose within any of these ranges, for therapeutic efficacy and/or symptomatic adjustment of the dosage to the patient to be treated. The compound will preferably be administered on a regimen of 1 to 4 times per day, preferably once or twice per day.

In one embodiment, CYT387 is administered orally once or twice daily in a unit tablet dose of 150 mg or 300 mg.

It will be understood that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The pharmaceutical composition may further comprise or be administered in combination with other therapeutically active compounds useful to treat the above mentioned pathological conditions. Selection of the appropriate agents for use in combination therapy may be made by one of ordinary skill in the art, according to conventional pharmaceutical principles.

For treatment of anemic subjects, CYT387 may be administered in combination with an anemia treatment drug, compound or modality selected from blood transfusion, iron supplements, erythropoietin or darbapoietin therapy, and the like. For treatment of subjects presenting with myeloproliferative disorders, CYT387 or a related compound can be administered in combination with thalidomide, lenalidomide, other JAK2 or JAK1/2 kinase inhibitors including those mentioned supra, in combination with hydroxyurea or with anagrelide, or in combination with bisphosphonates to decrease bone marrow fibrosis. As well, such patients can also undergo radiation therapy or allogeneic bone marrow transplantation, as part of the overall therapy that includes CYT387 or related compound dosing.

All publications mentioned in this specification are herein incorporated by reference. It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### EXAMPLES

CYT387 is an inhibitor of the kinase enzymes JAK1 and JAK2, which have been implicated in a family of hematological conditions known as myeloproliferative neoplasms, including myelofibrosis, and as well in numerous disorders including indications in hematology, oncology and inflammatory diseases. Myelofibrosis is a chronic debilitating disease in which a patient's bone marrow is replaced by scar tissue and for which treatment options are limited or unsatisfactory.

### Synthesis of CYT387

A mixture of 4-ethoxycarbonylphenyl boronic acid (23.11 g, 119 mmol), 2,4-dichloropyrimidine (16.90 g, 113 mmol), toluene (230 mL) and aqueous sodium carbonate (2 M, 56 mL) was stirred vigorously and nitrogen was bubbled through the suspension for 15 minutes. Tetrakis(triphenylphosphine)palladium[0] (2.61 g, 2.26 mmol) was added. Nitrogen was bubbled through for another 10 min, the mixture was heated to 100.° C., then at 75° C. overnight. The mixture was cooled, diluted with ethyl acetate (200 mL), water (100 mL) was added and the layers were separated. The aqueous layer was extracted with ethyl acetate (100 ml) and the two organic extracts were combined. The organics were washed with brine, filtered through sodium sulfate, concentrated, and the resultant solid was triturated with methanol (100 mL) and filtered. The solids were washed with methanol (2×30 mL) and air dried. This material was dissolved in acetonitrile (150 mL) and dichloromethane (200 mL), stirred with MP.TMT Pd-scavenging resin (Agronaut part number 800471) (7.5 g) over 2 days. The solution was filtered, the solids were washed with dichloromethane (2×100 mL), and the filtrate concentrated to give ethyl 4-(2-chloropyrimidin-4-yl)benzoate as an off-white solid (17.73 g, 60%)—additional washing with dichloromethane yielded a further 1.38 g and 0.5 g of product.

A mixture of ethyl 4-(2-chloropyrimidin-4-yl)benzoate (26.15 g, 99.7 mmol) and 4-morpholinoaniline (23.10 g, 129.6 mmol) was suspended in 1,4-dioxane (250 mL). p-Toluenesulfonic acid monohydrate (17.07 g, 89.73 mmol) was added. The mixture was heated at reflux for 40 h., cooled to ambient temperature, concentrated then the residue was partitioned between ethyl acetate and 1:1 saturated sodium bicarbonate/water (1 L total). The organic phase was washed with water (2×100 mL) and concentrated. The aqueous phase was extracted with dichloromethane (3×200 mL). The material which precipitated during this workup was collected by filtration and set aside. The liquid organics were combined, concentrated, triturated with methanol (200 mL) and filtered to yield additional yellow solid. The solids were combined, suspended in methanol (500 mL), allowed to stand overnight then sonicated and filtered. The solids were washed with methanol (2×50 mL) to give, after drying, ethyl 4-(2-(4-morpholinophenylamino)pyrimidin-4-yl)benzoate (35.39 g, 88%).

A solution of ethyl 4-(2-(4-morpholinophenylamino)pyrimidin-4-yl)benzoate (35.39 g, 87.6 mmol) in 3:1 methanol/tetrahydrofuran (350 mL) was treated with lithium hydroxide (4.41 g, 183.9 mmol) in water (90 mL). The mixture was heated at reflux for 2 h., cooled, concentrated and acidified with hydrochloric acid (2M, 92.5 mL, 185 mmol). The dark precipitate was filtered, washed with water, and dried under vacuum. The solid was ground to a powder with a mortar and pestle, triturated with methanol (500 mL) then filtered again to yield 4-(2-(4-morpholinophenylamino)pyrimidin-4-yl)benzoic acid as a muddy solid. This material was washed with ether, air dried overnight, and ground to a fine powder with mortar and pestle. On the basis of mass recovery (34.49 g) the yield was assumed to be quantitative.

To a suspension of 4-(2-(4-morpholinophenylamino)pyrimidin-4-yl)benzoic acid (theoretically 32.59 g, 86.6 mmol) in DMF (400 mL) was added triethylamine (72.4 mL, 519.6 mmol, 6 eq.) The mixture was sonicated to ensure dissolution. Aminoacetonitrile hydrochloride (16.02 g, 173.2 mmol) was added followed by N-hydroxybenzotriazole (anhydrous, 14.04 g, 103.8 mmol) and 1-ethyl-3-(dimethylaminopropyl)carbodiimide hydrochloride (19.92 g, 103.8 mmol). The suspension was stirred vigorously overnight. The solvent was evaporated under reduced pressure, the residue was diluted with 5% sodium bicarbonate (400 mL) and water (300 mL), giving a yellow solid, which was broken up and filtered. The solids were washed several times with 100 mL portions of water, triturated with hot methanol/dichloromethane (500 mL, 1:1), concentrated to a volume of approximately 300 mL), cooled and filtered. The solids were washed with cold methanol (3×100 mL), ether (200 mL) and hexane (200 mL) prior to drying to afford CYT387 (31.69 g, 88%). M.p. 238-243° C.

### CYT387 Clinical Results

A clinical trial was designed to assess the safety, tolerability, and pharmacokinetic behavior of CYT387 in a Phase I dose-escalation study in patients with high- or intermediate-risk primary myelofibrosis (PMF) and post-PV or post-essential thrombocythemia (ET) myelofibrosis. The secondary objective was evaluation of CYT387 's benefit to myelofibrosis patients. CYT387 was administered orally (in a capsule without excipients) once daily in 28-day cycles. For patients achieving less than a complete remission after 3 cycles of treatment, escalation was permitted to the highest tolerated dose in the absence of disease progression or unacceptable toxicity. Once dose-limiting toxicity (DLT) was identified, a dose-confirmation cohort initiated treatment at the maximum tolerated dose (MTD) and/or a lower clinically effective dose.

Results were obtained for 36 subjects enrolled in the 120 patient Phase I/II trial for which recruitment is ongoing. Of these, 18 patients were from the dose escalation phase, and 18 patients were from the subsequent dose confirmation phase. Twenty subjects (56%) were red cell transfusion-dependent at study entry. Prior treatment included other JAK inhibitors in ten patients (nine and one subjects with INCB018424 and TG101348, respectively) and pomalidomide in nine patients. The median treatment duration at publication was 15 weeks (range 4-38).

Potential predictors of CYT efficacy, in terms of anemia response and spleen response, emerged from this study, and are presented in Tables 1 and 2 below:

TABLE 1
Predictors of Anemia/Spleen Response - Clinical variables

| | Anemia response | | Spleen response | |
|---|---|---|---|---|
| | p-value | p-value | p-value | p-value |
| | (all | (transfusion | (all | (transfusion |
| | evaluable) | dependent) | evaluable) | dependent) |
| Variable | n = 42 | n = 33 | n = 53 | n = 27 |
| Age | NS | NS | NS | NS |
| Absence of | NS | NS | 0.03 | 0.01 |
| JAK2V617F | | | | |
| Karyotype | NS | NS | NS | NS |
| MF variant | NS | NS | NS | NS |
| DIPSS-Plus | NS | NS | NS | NS |
| category | | | | |
| Constitutional | NS | NS | NS | NS |
| symptoms at | | | | |
| baseline (pruritus, | | | | |
| bone pain, fever) | | | | |
| WBC or Platelet | NS | NS | NS | NS |
| count | | | | |
| Smaller spleen size | NS | NS | 0.0004 | 0.04 |
| Presence of night | 0.09 | 0.03 | NS | NS |
| sweats at baseline | | | | |
| Lower percentage of | 0.08 | 0.03 | 0.04 | 0.07 |
| circulating blasts | | | | |
| Prior cytoreductive | NS | NS | NS | NS |
| therapy (incl. HU) | | | | |
| Prior pomalidomide | NS | NS | NS | NS |
| therapy | | | | |
| Prior lenalidomide | 0.008 | 0.001 | NS | NS |
| therapy | | | | |
| Prior thalidomide | 0.04 | 0.03 | NS | |
| therapy | | | | |
| Prior JAK inhibitor | NS | NS | 0.01 | 0.006 |
| (INCB018424 or | | | | |
| TG101348) | | | | |
| Prior ESA therapy | NS | NS | 0.02 | NS |
| IWG spleen | NS | NS | n/a | NS |
| response | | | | |
| ‘First-dose’ effect | NS | NS | NS | NS |

TABLE 2
Predictors of Anemia/Spleen Response - Markers

| | Anemia response | | Spleen response | |
|---|---|---|---|---|
| | | Transfusion- | All | Transfusion- |
| | All evaluable | dependent | evaluable | dependent |
| Cytokine | (n = 42) | (n = 33) | (n = 53) | (n = 27) |
| Increased EGF | NS | NS | 0.01 | 0.01 |
| Increased TNF-α | NS | NS | 0.01 | 0.05 |
| Increased | NS | NS | 0.03 | 0.08 |
| G-CSF | | | | |
| Increased IFN-α | NS | NS | 0.02 | 0.03 |
| Increased MIP-1β | NS | NS | 0.006 | 0.004 |
| Increased HGF | NS | NS | NS | 0.01 |
| Increased MIG | NS | NS | NS | 0.01 |
| Increased VEGF | NS | NS | 0.03 | 0.008 |
| Decreased | 0.006 | 0.006 | NS | NS |
| Eotaxin | | | | |

### Efficacy Results:

Anemia Response: The total anemia response rate was 63%. Of 22 subjects who were evaluable for anemia response (baseline Hgb<10 g/dL or red cell transfusion-dependent), nine subjects (41%) had achieved “Clinical Improvement (CI)” as per the International Working Group for Myeloproliferative Neoplasms Research and Treatment (IWG-MRT) criteria, including two of four subjects who were previously treated with INCB018424. An additional five subjects experienced a >50% reduction in transfusion requirement.

Spleen Size Reduction: Twenty nine (97%) of the 30 evaluable subjects who had splenomegaly at baseline (median 20 cm; range 10-32 cm) had some degree of spleen size reduction (median 9 cm; range 2-18 cm). Eleven (37%) patients have achieved a minimum 50% decrease in palpable spleen size, thus qualifying them for a CI per IWG-MRT criteria, including three out of eight subjects (38%) who were previously treated with INCB018424.

Constitutional symptoms: The proportion of patients with the following symptoms at baseline was as follows: fatigue (97%), pruritus (22%), night sweats (38%), cough (13%), bone pain (28%), and fever (16%). At last follow up, improvement (complete resolution; CR) in these symptoms was reported by 68% (16% CR), 86% (57% CR), 83% (75% CR), 75% (50% CR), 78% (44% CR), and 100% (100% CR), respectively.

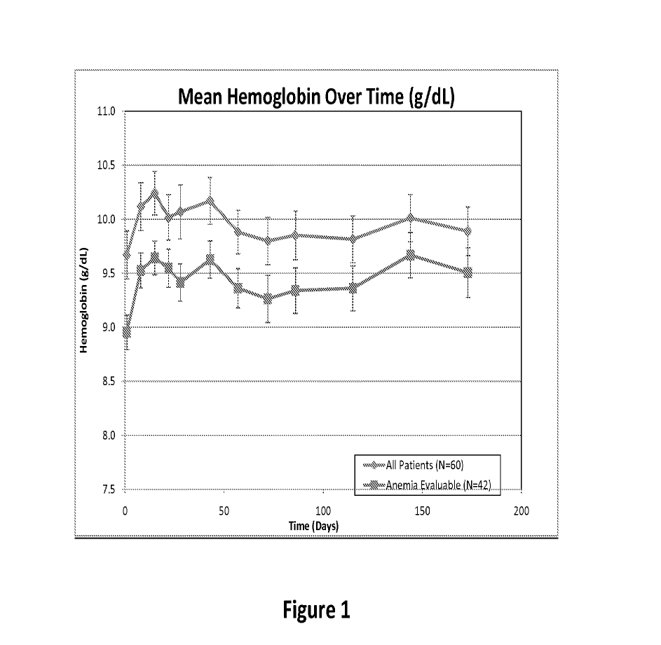

Further analysis has revealed that subjects responded very favourably to CYT387 in terms of their hemoglobin levels/anemia response. FIG. 1 shows the mean hemoglobins for all of the first 60 patients enrolled in the study (up to 6 months or more). The graph shows the mean hemoglobins over time from baseline, when CYT387 was first dosed. There is also a line for “anemia evaluable” patients which are patients who are considered anemic at baseline by IWG criteria. That is they have a baseline hgb of <10 g/dL or are transfusion dependent at baseline.

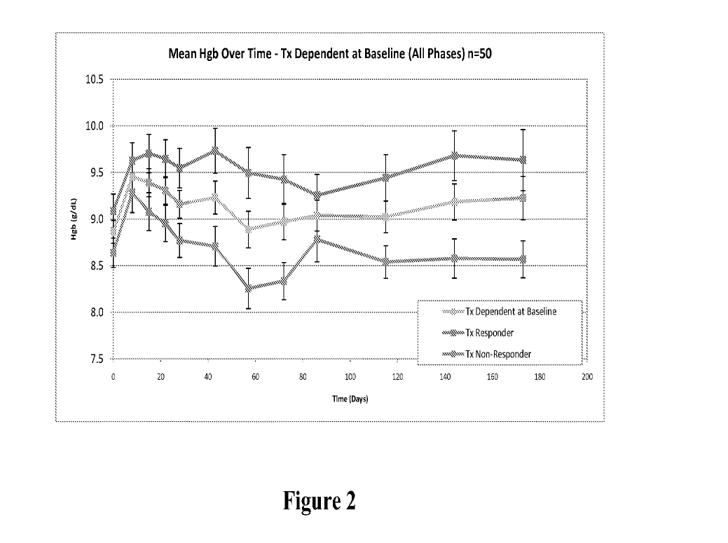

FIG. 2 illustrates that the patients who respond to CYT387 demonstrate a modest initial increase in hemoglobin that is sustained even though they are no longer receiving transfusions of RBC. These responders, who are no longer being transfused, maintain a mean hemoglobin level that is higher than the mean hemoglobin level of the non-responders who continue to be transfused.

As shown, administration of CYT387 provides a clear and sustained improvement in hemoglobin levels in these patients.

Still further analysis revealed the following:

### Interim Response by Diagnosis

| Response | |
|---|---|
| (n = 165) | Total (n = 165) |
| PMF (n %) | 105/165 (63.6%) |
| Spleen Response/Evaluable | 22/82 (26.8%) |
| Anemia Response/Evaluable | 30/66 (45.5%) |
| PPV (n %) | 36/165 (21.8%) |
| Spleen Response/Evaluable | 11/33 (33.3%) |
| Anemia Response/Evaluable | 6/14 (42.9%) |
| PET (n %) | 24/165 (14.6%) |
| Spleen Response/Evaluable | 7/19 (36.8%) |
| Anemia Response/Evaluable | 10/18 (55.6%) |

### Interim Response in Previously Treated Patients

| Population | Responders |
|---|---|
| Previous Treatment with INCB018424 | Anemia: 4/7 = 57.1% |
| | Spleen: 3/10 = 30% |
| Previous Treatment with TG-101348 | Anemia: 3/4 = 75% |
| | Spleen: 1/4 = 25% |
| Previous Treatment with Unspecified JAK2 | Anemia: 2/3 = 66.7% |
| | Spleen: 1/3 = 33.3% |
| Previous Treatment with any JAK2 | Anemia: 9/14 = 64.3% |
| | Spleen: 5/17 = 29.4% |
| Previous Treatment with IMids | Anemia: 9/14 = 64.3% |
| (Pomalidomide, Thalidomide, Revlimid) | Spleen: 6/11 = 54.5% |

### Interim Response by Initial Dose—Anemia Response

| | Initial Dose | | |
|---|---|---|---|
| | 150 QD | 300 QD | 150 BID |
| Evaluable Pts receiving starting dose | 32 | 39 | 21 |
| Responders | 16 | 21 | 7 |
| % | 50% | 54% | 33%* |

### Toxicity Results:

To date, 36 subjects were evaluable for toxicity. At the highest dose level (400 mg/day), two of six subjects experienced dose limiting toxicity (DLT) (one each with asymptomatic grade three hyperlipasemia and grade three headache that were reversible upon holding drug); consequently, the maximum tolerated dose (MTD) was declared at 300 mg/day. In the dose-confirmation phase, subjects were started at one of two dose levels that were deemed clinically effective: 150 mg/day (n=15) and 300 mg/day (n=3). Thirty-five subjects were on active therapy at publication of the abstract: 100 mg/day (n=2), 150 mg/day (n=20), 300 mg/day (n=10), and 400 mg/day (n=3).

CYT387 was well tolerated. No grade 4 non-hematological toxicities were observed. Grade 3 non-hematologic adverse events were infrequent and included increased transaminases (n=2), increased alkaline phosphatase (n=2), headache/head pressure (n=2), increased lipase (n=1), and QTc prolongation (n=1). Thirteen (36%) subjects experienced “first-dose effect” characterized by grade 1 lightheadedness and hypotension; this phenomenon was self-limited and generally resolved within 3-4 hours with rare recurrence. Grade 3/4 thrombocytopenia was seen in eight (22%) subjects, and treatment-emergent grade 3 anemia was seen in one subject only (3%). Treatment-emergent grade 3/4 neutropenia was not observed.

1. A method for treating a subject experiencing anemia, comprising administering to the subject an amount of N-(cyanomethyl)-4-[2-[[4-(4-morpholinyl)phenyl]amino]-4-pyrimidinyl]-benzamide [CYT387 ] or a related compound effective to maintain or elevate the hemoglobin level.

2. The method according to claim 1, wherein the subject experiencing anemia is afflicted with a myeloproliferative disorder.

3. The method according to claim 2, wherein the subject experiencing anemia is afflicted with primary myelofibrosis.

4. The method according to claim 3, wherein the subject experiencing anemia is afflicted with primary myelofibrosis secondary to polycythemia vera.

5. The method according to claim 3, wherein the subject experiencing anemia is afflicted with primary myelofibrosis secondary to essential thrombocythemia.

6. The method according to claim 2, wherein the subject experiencing anemia is afflicted with myelodisplastic syndrome.

7. The method according to any one of claims 1-6, wherein the compound is CYT387.

8. The method according to claim 7, wherein the compound is the dihydrochloride salt of CYT387.

9. The method according to any one of claims 1-8, wherein the anemic subject is not afflicted with a myeloproliferative disorder.

10. The use of CYT387 to treat anemia.

11. An article of manufacture, comprising a container comprising CYT387 in an amount useful to treat anemia and, associated with said container, a label indicating treatment of a subject afflicted with anemia.

12. A kit comprising a container comprising CYT387 in an amount useful to treat anemia and, in association therewith, printed instruction teaching a method for said treatment.

13. An improved method for treating a subject having, or at risk for, a medical condition for which a JAK inhibitor is indicated, the method comprising the steps of (1) selecting, for treatment, a subject that meets at least one of the following criteria:
(i) prior therapy with a drug selected from thalidomide, lenalidomide, pomalidomide and a JAK inhibitor other than CYT387;
(ii) a clinical criterion selected from one or both of (1) larger spleen size and (2) lower percentage of circulating blasts;
(iii) a biochemical marker criterion selected from one or more of (1) an increased level of at least one protein selected from EGF, TNF-α, G-CSF, IFN-α, MIP-1β, HGF, MIG, and VEGF; (2) a decreased level of eotaxin; and (3) an altered level of at least one protein selected from EPO, hepcidin and BMP-2;
and then (2) administering to the selected subject a treatment-effective amount of CYT387, whereby the treated subject exhibits an anemia response and/or a spleen response that is improved relative to a subject not meeting any of said criteria.

14. The method according to claim 13, wherein the indication for which the JAK inhibitor is indicated is a myeloproliferative neoplasm.

15. The method according to claim 14, wherein the myeloproliferative neoplasm is primary myelofibrosis.

16. The method according to claim 13, wherein the myeloproliferative neoplasm is primary myelofibrosis secondary to polycythemia vera.

17. The method according to claim 13, wherein the myeloproliferative neoplasm is primary myelofibrosis secondary to essential thrombocythemia.

18. The method according to claims 13-17, comprising the preliminary step of screening said subject or a biological sample obtained therefrom to assess at least one of said criteria.

19. The method according to claim 18, wherein a biological sample obtained from said subject is screened to assess the level of at least one of said biochemical markers.

20. An article of manufacture, comprising container comprising CYT387 in an amount useful to treat MPN and, associated with said container, a label indicating treatment of a subject identified according to claim 13.

21. A kit comprising a container comprising CYT387 in an amount useful to treat MPN and, in association therewith, printed instruction teaching a method according to claim 13.
